(19) United States
(12) Patent Application Publication
(10) Pub. No.: US 2004/0224880 A1
(43) Pub. Date: Nov. 11, 2004

(54) Regulation of gastric acid secretion by inwardly rectifying k+ channels

(75) Inventor: Susan J. Hagen, Acton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Beth Israel Deaconess Medical Center, Inc., Boston, MA (US)

(21) Appl. No.: 10/485,003

(22) PCT Filed: Jul. 26, 2002
(86) PCT No.: PCT/US02/23977

Related U.S. Application Data
(60) Provisional application No. 60/308,387, filed on Jul. 27, 2001.

(51) Int. Cl. A61K 38/17

(52) U.S. Cl. 514/12

(57) ABSTRACT

This invention relates to a method to treat an individual with a disorder associated with gastric acid secretion mediated by an apical, inwardly rectifying K+ channel. The disorder associated with gastric acid secretion is selected from gastric ulcers, duodenal ulcers, gastritis, duodenitis, reflux esophagitis or Helicobacter pylori infection. The apical, inwardly rectifying K+ channel preferably comprises Kir4.1. The agent to be administered is an antagonist of the apical, inwardly rectifying K+ channel such as ammonium chloride.

### RELATED APPLICATION

This application claims the benefit of U.S. Provisional Application No. 60/308,387, filed Jul. 27, 2001.

The entire teachings of the above application is incorporated herein by reference.

### GOVERNMENT SUPPORT

The invention was supported, in whole or in part, the National Institute of Diabetes and Digestive and Kidney Diseases, grant number R01 DK-15681. The Government has certain rights in the invention.

### BACKGROUND OF THE INVENTION

Inhibition of gastric acid secretion is a multi-billion dollar business in the U.S.A. and abroad. Acid secretion occurs in the stomach by activity of an H, K-ATPase located within the apical membrane of stimulated parietal cells. Two of the drugs currently available for acid inhibition are H2 blockers (cimetidine) which block activity of the histamine H2 receptor and proton pump inhibitors (PPI's) which directly block activity of the H, K-ATPase. In addition to numerous basolateral K⁺ channels that recycle K⁺ imported by the Na, K-ATPase, parietal cells are thought the possess an apical K⁺ channel(s) that is (are) active only during acid secretion. Apical K⁺ channel activity could facilitate the movement of cytoplasmic K⁺ into the gastric lumen and this K⁺ would then be recycled by the H, K-ATPase activity during acid secretion. However, the relationship of any apical K⁺ channel with regulation of acid secretion has still to be determined, and no drug is currently available to block apical K⁺ channel activity and acid secretion.

### SUMMARY OF THE INVENTION

The present invention relates to a method to regulate acid secretion mediated by an apical, inwardly rectifying K⁺ channel in gastrointestinal cells by contacting the cells with an agent that regulates the activity of the apical, inwardly rectifying K⁺ channel in gastrointestinal cells and thus regulating or modifying the acid secretion which is mediated by the apical K⁺ channel. In one embodiment of the present invention, the acid secretion is stimulated. The gastrointestinal cells described herein, which contain the apical, inwardly rectifying K⁺ channel, are typically oxyntic and parietal cells. In another embodiment of the present invention, the apical, inwardly rectifying K⁺ channel comprises the protein Kir 4.1. Furthermore, other proteins can also be associated with the apical, inwardly rectifying K⁺ channel, for example Kir 5.1, Kir 4.2 or Kir1.1 proteins. In addition, the apical, inwardly rectifying K⁺ channel may be homomeric or heteromeric for these proteins. In another embodiment, acid secretion mediated by the apical, inwardly rectifying K⁺ channel in gastrointestinal cells is inhibited by the administration of an antagonist which can be a small molecule inhibitor or a weak base inhibitor wherein acid secretion is reduced or inhibited.

This invention also pertains to a method to regulate the activity or function of an apical, inwardly rectifying K⁺ channel, wherein the activity or function of the apical, inwardly rectifying K⁺ channel is associated with acid secretion. In one embodiment, the method comprises contacting the apical, inwardly rectifying K⁺ channel with an agent that alters the activity or function of the apical, inwardly rectifying K⁺ channel, thereby regulating or modulating the activity or function of the apical, inwardly rectifying K⁺ channel. In another embodiment of this invention, the apical, inwardly rectifying K⁺ channel comprises Kir4.1. In another preferred embodiment, the activity or function of the apical, inwardly rectifying K⁺ channel is inhibited. Inhibition of the apical, inwardly rectifying K⁺ channel can be mediated by the administration of an antagonist of the apical, inwardly rectifying K⁺ channel. Preferably, the antagonist is selected from the group consisting of small molecule inhibitors and weak base inhibitors.

Another embodiment of this invention pertains to a method to screen for an agent or agents that regulate acid secretion comprising assaying the quantity of acid secretion, mediated by an apical, inwardly rectifying K⁺ channel, from parietal cell or oxyntic cells stimulated to secrete acid in the presence and absence of the agent to be tested. Reduced acid secretion in the presence of the agent being tested indicates that the agent is an antagonist of acid secretion.

This invention also relates to a method to treat an individual with a disorder associated with gastric acid secretion mediated by an apical, inwardly rectifying K⁺ channel, comprising the steps of administering a therapeutically effective amount of an agent that regulates or modifies the activity of the apical, inwardly rectifying K⁺ channel to the individual. In one embodiment, the disorder associated with gastric acid secretion mediated by an apical, inwardly rectifying K⁺ channel is selected from the group consisting of gastric ulcers, duodenal ulcers, gastritis, duodenitis, and reflux esophagitis. In a further embodiment, the disorder associated with gastric acid secretion mediated by an apical, inwardly rectifying K⁺ channel is associated with Helicobacter pylori infection. In another embodiment, the apical, inwardly rectifying K⁺ channel comprises Kir4.1. Furthermore, in another embodiment, the agent to be administered to an individual with a disorder associated with gastric acid secretion, mediated by an apical, inwardly rectifying K⁺ channel, is an antagonist of the apical, inwardly rectifying K⁺ channel. The preferred antagonist is selected from the group consisting of a small molecule inhibitor and a weak base inhibitor.

### BRIEF DESCRIPTION OF THE DRAWINGS

FIG. 1A Effect of basolateral NH₄Cl on stimulated acid secretion. In tissues stimulated with histamine/carbachol for 90 min, addition of 10 mM NH₄Cl to the nutrient buffer completely abolished acid secretion within 30 min. Removal of NH₄Cl was followed by a complete recovery of acid secretion within 30 min. Data were obtained from 21 experiments and are expressed as the mean±SEM.

FIG. 1B Electrophysiology in the bullfrog gastric mucosa. In tissues stimulated with histamine/carbachol for 90 min, addition of 10 mM NH₄Cl to the nutrient buffer and resulted in an increase in resistance (R) within 30 min. Removal of NH₄Cl was followed by a complete recovery of R within 30 min. Data were obtained from 21 experiments and are expressed as the mean±SEM.

FIG. 1C Electrophysiology in the bullfrog gastric mucosa. In tissues stimulated with histamine/carbachol for 90 min, addition of 10 mM NH₄Cl to the nutrient buffer and resulted in an increase in potential difference (PD) within 30 min. Removal of NH₄Cl was followed by a complete recovery of PD within 30 min. Data were obtained from 21 experiments and are expressed as the mean±SEM.

FIG. 2 Electron micrograph of a gastric gland from tissues stimulated with histamine/carbachol for 90 min and then incubated for 30 min with 10 mM NH₄Cl. Note that oxynticopeptic cells (OP) that line the gastric gland have an apical secretory surface that is in the stimulated configuration (arrowheads) with many surface folds projecting into the gland lumen (L). LP, lamina propria. Original magnification: ×4,600; bar=5 μm.

FIG. 3(A) Effect of methylamine on acid secretion in the bullfrog gastric mucosa. In tissues stimulated with histamine/carbachol, 50 mM methylamine (MA) was required to completely inhibit acid secretion within 30 min. Removal of methylamine was followed by partial recovery of acid secretion. Data were obtained from 7 experiments and are expressed as the mean±SEM.

FIG. 3(B) Effect of imidazole on acid secretion in the bullfrog gastric mucosa. In tissues stimulated with histamine/carbachol, imidazole (Iz) had no effect on acid secretion when used at a concentration similar to that of NH₃ in FIG. 1 (100 μM). Tissues treated with imidazole were then challenged with 10 mM NH₄Cl, acid secretion was inhibited completely within 30 min. Data were obtained from 7 experiments and are expressed as the mean±SEM.

FIG. 4 Flux (serosal to mucosal) of methylamine in stimulated and inhibited tissues. Methylamine (50 mM), containing 5 μCi of [¹⁴C]-methylamine, was added to the nutrient solution of tissues stimulated with histamine/carbachol or inhibited with cimetidine. Samples were taken every 15 min and the concentration of methylamine determined from counts that were collected by liquid scintillation. The flux rate of methylamine in inhibited tissues was 0.601±0.114 μM/h cm² and 2.25±0.088 μM cm² in stimulated tissues. Data were obtained from 7 experiments and are expressed as mean±SEM.

FIG. 5A Effect of barium on acid secretion in the presence of NH₄Cl. When 0.5 and 1 mM barium were added to tissues stimulated with histamine/carbachol, acid was not different from that of control tissues. In contrast, acid secretion gradually declined in tissues incubated with 5 mM barium and was significantly (P=0.0085) different from control tissues after 1 hr (150 min). When 10 mM NH₄Cl was added to tissues treated with barium, acid secretion was abolished within 30 min. Data were obtained from 6-8 experiments and are expressed as mean±SEM.

FIG. 5B Effect of barium on acid secretion in the presence of NH₄Cl. Tissue resistance (R) increased significantly (P<0.002) in the presence of 0.5, 1 and 5 mM barium. When 10 mM NH₄Cl was added to tissues treated with 0.5 and 1 mM barium, R increased similar to that of control tissues. Data were obtained from 6-8 experiments and are expressed as mean±SEM.

FIG. 5C Effect of barium on acid secretion in the presence of NH₄Cl. The potential difference (PD) of tissues stimulated with histamine/carbachol was unaffected by barium. When 10 mM NH₄Cl was added to tissues treated with barium, PD increased in all groups. Data were obtained from 6-8 experiments and are expressed as mean±SEM.

FIG. 6A Effects of tetraethylammonium (TEA) on acid secretion in the presence of NH₄Cl. In tissues stimulated with histamine/carbachol, TEA had no effect on stimulated acid secretion. When tissues were further incubated with 10 mM NH₄Cl, acid secretion was abolished within 30 min. R increased in all tissues incubated with NH₄Cl. Control tissues were incubated with an equal concentration of DMSO (0.1%). Data were obtained from 7 experiments and are expressed as the mean±SEM.

FIG. 6B Effects of tolbutamide and clotrimazole on acid secretion in the presence of NH₄Cl. In tissues stimulated with histamine/carbachol, tolbutamide and clotrimazole had no effect on stimulated acid secretion. When tissues were further incubated with 10 mM NH₄Cl, acid secretion was abolished within 30 min. Control tissues were incubated with an equal concentration of DMSO (0.1%). Data were obtained from 7 experiments and are expressed as the mean±SEM.

FIG. 6C Effects of tetraethylammonium (TEA) on resistance (R) in the presence of NH₄Cl. In tissues stimulated with histamine/carbachol, TEA had no effect on R. When tissues were further incubated with 10 mM NH₄Cl, R increased in all tissues. Control tissues were incubated with an equal concentration of DMSO (0.1%). Data were obtained from 7 experiments and are expressed as the mean±SEM.

FIG. 7A Effect of inhibition of basolateral transporter activity on acid secretion in the presence of NH₄Cl. When tissues stimulated with histamine/carbachol were incubated with 100 μM bumetamide, 1 μM amiloride, or 1 mM oubain, the rate of acid secretion was unchanged during the initial 60 minutes. When 10 mM NH₄Cl was added to tissues incubated with bumetamide, amiloride or oubain, acid secretion declined within 30 min. Data were obtained from 7 experiments and are expressed as the mean±SEM.

FIG. 7B Effect of inhibition of basolateral transporter activity on acid secretion in the presence of NH₄Cl. When tissues stimulated with histamine/carbachol were incubated with 0.3 mM DIDS, the rate of acid secretion in control tissues declined in 30 min to approximately one half of the initial rate and then remained at that rate for the next 30 min. When 10 mM NH₄Cl was added to tissues incubated with DIDS, acid secretion declined within 30 min. Data were obtained from 7 experiments and are expressed as the mean±SEM.

FIG. 8A Effects of ATP-depletion on acid secretion in the frog gastric mucosa and of NH₄Cl on ATP-depletion in rabbit gastric glands. When tissues were stimulated with histamine/carbachol and then incubated with 2 mM potassium cyanide (KCN), the rate of acid secretion decreased within 30 min. Potassium chloride (KCl) added at 2 mM had little effect on acid secretion. Data were obtained from 6 experiments and were expressed as the mean±SEM.

FIG. 8B Effects of ATP-depletion on acid secretion in the frog gastric mucosa and of NH₄Cl on ATP-depletion in rabbit gastric glands. The concentration of ATP was measured in rabbit gastric glands that were incubated for 30 min with gland buffer (control), 10 mM NH₄Cl, or 2 mM KCN. A statistically significant reduction in the concentration of ATP was found in glands incubated with NH₄Cl (P=0.037) or with KCN (P<0.0001). Data were obtained from 5 experiments and are expressed as the mean±SEM.

FIG. 9 Effect of basolateral NH₄Cl on the resistance (R) of tissues inhibited with cimetidine. Paired tissues were inhibited with cimetidine until acid secretion was 0 μEq/hr cm² and then incubated with either 10 mM NH₄Cl or 10 mM NaCl for 60 min. Note that NH₄Cl does not cause an increase in R in cimetidine-inhibited tissues as it does in tissues stimulated with histamine/carbachol in FIG. 1B. Data were obtained from 6 experiments and are expressed as the mean±SEM. Error bars are small and found within the boundary of each circle.

### DETAILED DESCRIPTION OF THE INVENTION

Hypochlordria and inflammation are hallmarks of early infection with Helicobacter pylori (HP), a pathogenic bacterium that colonizes the gastric antrum and fundus and leads to chronic-active gastritis and peptic ulcer disease. During early infection with HP, acid secretion may be suppressed by one or more products of HP or by inflammatory mediators that act directly on parietal cells (Calam et al. (1997) Gastroenterology) 113: S43-S49; Mobley (1997) Gastroenterology 113: S21-S28). Among the various factors liberated by HP (Calam et al. (1997) Gastroenterology 113: S43-S49), urease may be the most important because it leads to the production of large quantities of ammonia that can affect cell function and viability (Murakami et al. (1993) Gastroenterology 105: 1710-1715; Ricci et al. (1993) Eur. J. Gastroenterol. Hepatol. 5: 687-694; Triebling et al. (1991) Dig. Dis. Sci. 36: 1089-1096; Tsujii et al. (1992) Gastroenterology 102: 1881-1888; Yanaka et al. (1993) Am. J. Physiol. 265: G277-G288). Hypochlorhydria can also occur during chronic renal or hepatic failure, or after renal transplantation, when circulating levels of blood ammonia are extremely high (Gingell et al. (1968) Brit. Med. J 4: 424-426; Mathur and Agrawal (1973) J. Indian Med. Assoc. 60: 288-291; Muto, S et al. (1985) Nephron 40: 143-148).

Ammonia (NH₃) is a small molecule of 17 Da that acts as a weak base and is at equilibrium with its protonated form (NH₄⁺) at a pKₐ of 9.233. Thus, most of the NH₃ produced by HP will exist as NH₄⁺in the acidic environment of the gastric lumen. The apical (or luminal) surface of both gastric parietal and chief cells is relatively impermeable to NH₃ and NH₄⁺ (Boron et al. (1994) J. Exp. Biol. 196: 347-360), suggesting that ammonia formed in the gastric lumen during HP infection would be unable to alter gastric epithelial cell function. However, luminal administration of 115 mM NH₄Cl at pH 8 (2.7 mM NH₃) results in a significant decrement in tissue resistance and an increase in H⁺ back-diffusion that does not occur when the same concentration of NH₄Cl is used at pH 5 (3 μM NH₃) (Yanaka et al. (1993) Am. J. Physiol. 265: G277-G288), indicating that luminal NH₃ can cause an increase in the paracellular permeability of the gastric mucosa. In addition, vacuolating cytotoxin produced in vacA⁺ Helicobacter pylori bacteria increases the paracellular permeability of cultured gastric (GSM06), colonic (T84), and kidney (MDCK) cells (Papini et al. (1998) J. Clin. Invest. 102: 813-820; Yanaka et al. (1999) Gastroenterology 116: A362). Thus, both ammonia and vacuolating cytotoxin may facilitate the movement of gastric luminal contents, including ammonia to the serosal compartment during infection.

Whereas the apical surface of gastric epithelial cells is impermeable to ammonia, NH₃ is freely permeable across the basolateral cell membrane and NH₄+can enter cells via cation channels, pumps or exchange proteins (Boron et al. (1994) J. Exp. Biol. 196: 347-360). Basolateral exposure of the bullfrog gastric mucosa to 30 mM NH₄Cl at pH 7.2 (0.47 mM NH₃ and 29.53 mM NH₄⁺) rapidly and completely inhibits acid secretion and accelerates H⁺ back-diffusion while it alkalinizes, induces depolarization, and decreases electrogenic Cl⁻ secretion in bullfrog oxynticopeptic cells (Yanaka et al. (1993) Am. J. Physiol. 265: G277-G288).

Basolateral exposure of gastric epithelial cells to NH₃/NH₄⁺ also allows these substances to interact directly with basolateral ion channels, transporters, and exchangers. Although little is known about the effects of NH₃ and/or NH₄⁺ on basolateral ion transporters/exchangers in the gastric mucosa, it was suggested by Yanaka et al. ((1993) Am. J. Physiol. 265: G277-G288), that NH₄Cl may block basolateral K⁺ channel activity. This conclusion was derived from the observation that high K+(15 mM) in the nutrient solution attenuated the decrease in both PD and R that occurred after exposure to 30 mM nutrient NH₄Cl (Yanaka et al. (1993) Am. J. Physiol. 265: G277-G288). In addition, partial blockade of K⁺ channels with 0.2 mM barium (Ba²⁺) accelerated the rapid decrease in PD in the presence of NH₄Cl (Yanaka et al. (1993) Am. J. Physiol. 265: G277-G288). Frog oxyntic cells have Ca²⁺-activated, 61 ps, voltage-independent K⁺ channels that are sensitive to Ba²⁺ and cAMP-activated, 30 ps, Ca²⁺-insensitive K⁺ channels (Mieno et al. (1994) J. Gastroenterol 29 (suppl VII): 55-58). Other intracellular targets of NH₃/NH₄⁺ may be Cl⁻/HCO₃⁻ exchange (Yanaka et al. (1993) Am. J. Physiol. 265: G277-G288) or inhibition of ATP production by mitochondria (Tsujii et al. (1992) Gastroenterology 102: 1881-1888).

In contrast, the present invention demonstrates that blockade of basolateral K⁺ channel activity does not greatly influence the rate of stimulated acid secretion in the bullfrog gastric mucosa and thus cannot be the primary site of action of NH₄Cl. In addition, NH₄Cl does not inhibit acid secretion by altering basolateral Na⁺/H⁺ exchange, Na⁻—K⁺²Cl⁻ co-transport, the Na⁺/K⁺-ATPase, Cl⁻/HCO₃⁻ exchange, or Na⁺/HCO₃⁻ co-transport. Furthermore, NH₄Cl does not inhibit acid secretion by neutralizing secreted H⁺ in the gastric lumen or by its effects as a weak base on cytosolic and endosomal pH. Although ATP-depletion occurs in the presence of NH₄Cl, the magnitude of ATP-depletion is not sufficient to inhibit stimulated acid secretion.

This invention demonstrates that NH₄Cl effects the resistance of stimulated but not inhibited tissues, therefore NH₄Cl specifically targets stimulated acid secretion. Furthermore, this invention specifically relates to NH₄Cl blocking the activity of an apical K⁺ channel in bullfrog oxynticopeptic cells and that apical K⁺ channel activity is important for the regulation of acid secretion in the bullfrog gastric mucosa. The present invention shows that basolateral exposure of gastric oxyntic cells to as little as 92.5 μM ammonia (NH₃), present in 10 mM NH₄Cl at pH 7.2, completely inhibits acid secretion within 30 min. Similar concentrations of ammonia could permeate the gastric mucosa during acute infection with Helicobacter pylori through defects in the epithelium, or circulate in the blood of patients with chronic renal failure or hepatic disease. This inhibition of acid secretion with NH₄Cl is more than 3 times faster than with other acid inhibitors such as cimetidine (H2 receptor agonist) or omeprazole (proton pump inhibitor). Since NH₄Cl inhibits acid secretion after stimulation with histamine/carbachol or forskolin, it is likely that NH₄Cl acts at a site distal to H2 receptor binding and to the activation of adenylate cyclase in the cAMP-signaling cascade. Inhibition of acid secretion by NH₄Cl was accompanied by an increase in PD and R nearly identical in magnitude to the increase in PD and R that occurred during inhibition of acid secretion by either cimetidine or omeprazole.

Whereas the NH₄Cl-induced inhibition of acid secretion in the present invention was completely reversed after withdrawal of NH₄Cl, Yanaka et al. ((1993) Am. J. Physiol. 265: G277-G288) showed only minimal recovery after withdrawal of NH₄Cl. It is reasonable to believe from the data presented here that the ability of acid secretion to recover fully after an acute challenge with NH₄Cl is concentration-dependent. That the morphology of oxyntic cells remained stimulated despite the absence of acid secretion accounts for the ability of these cells to regain nearly normal rates of acid secretion rapidly after the withdrawal of NH₄Cl from the nutrient solution; reassembly of the apical surface takes nearly 2 hours from the inhibited configuration in frog gastric mucosa (Hagen, et al. (1994) Cell Tissue Res 275: 255-267).

The way in which NH₃ or NH₄⁺ enters the basolateral membrane of gastric oxyntic cells is not known. However, the basolateral membrane of gastric oxyntic cells possess multiple permeability pathways for Na⁺ and K⁺ (Na⁺/H⁺ exchange, Na⁺/HCO₃⁻ cotransport, Na⁺—K⁺—2Cl⁻-cotransport, Na⁺/K⁺-ATPase) which could transport NH₄⁺ into the cell. This invention demonstrates that basolateral ion transporters do not facilitate the rapid movement of NH₄⁺ into gastric oxyntic cells because blockade of basolateral ion transport activity did not mitigate the effect of NH₄Cl on acid secretion. It is reasonable to believe from the information presented here that NH₃ enters cells at the basolateral membrane by passive diffusion and then is rapidly converted to NH₄⁺ in the cytoplasm at neutral pH. The results presented here differ from those obtained in the kidney where NH₄⁺ is moved rapidly into cells by renal Na⁺/K⁺-ATPase or by Na⁺—K⁺—2Cl⁻ co-transporter activity depending on cell-type (Knepper et al. (1989) Physiol. Rev. 69: 179-249).

Several lines of evidence from the present invention support the conclusion that NH₄Cl does not block acid secretion by blockade of basolateral ion transport activity. Inhibition of Na⁺/H⁺ exchange with amiloride, Na⁺/K⁺-ATPase with oubain, or Na⁺—K⁺—2Cl⁻ co-transport with bumetamide for 30 min has little effect on stimulated acid secretion in the frog gastric mucosa. Thus, even if NH₄Cl inhibited the activity of one of these three exchangers, there would be no demonstrable effect on acid secretion. In contrast, the present invention shows that acid secretion in the frog gastric mucosa was partially, but not completely, inhibited by blockade of Na⁺/HCO₃⁻ co-transport and/or Cl⁻/HCO₃⁻ exchange with DIDS. That acid secretion can be inhibited by blockade of Na⁺/HCO₃⁻ co-transport and/or Cl⁻/HCO₃⁻ exchange suggests that NH₄Cl could reduce acid secretion, in part, by blockade of one or both of these basolateral transporters. Against this possibility is the finding that the addition of NH₄Cl to tissues incubated with DIDS immediately reduced acid secretion to zero, suggesting that NH₄Cl and DIDS act at different sites. Thus, it is unlikely that NH₄Cl inhibits acid secretion by blocking Na⁺/HCO₃ co-transport and/or Cl⁻/HCO₃⁻ exchange in gastric oxyntic cells.

Imidazole (Iz) can be used to mimic the effects of ammonia (NH₃) as a weak base. Like NH₃, an equimolar concentration of Iz should result in a similar amount of cytosolic alkalinization and neutralization of acidic intracellular compartments due to its ability to easily permeate cell membranes (Megraud et al. (1992) Infect. Immun. 60: 1858-1863). However, when acid secretion was measured for 3 hr in the presence of 200 μM nutrient Iz at pH 7.1 (100 μM Iz versus 92.3 μM NH₃), the rate of acid secretion was not significantly different from that of control tissues. Furthermore, acid secretion in 3 hr was inhibited only slightly from 6.6 μEq/hr cm² to 4.03 μEq/hr cm² in the presence of 10 mM nutrient Iz at pH 7.1 (5 mM Iz). That 10 mM nutrient NH₄Cl inhibited acid secretion immediately in the presence of either 200 μM or 5 mM imidazole at pH 7.1, suggests that the action of ammonia and imidazole are different.

Methylamine, a C₁ primary alkylamine that is larger and more lipophilic than ammonia (C₀ primary alkylamine), can be used to test the effects of related primary alkylamines on acid secretion. Methylamine rapidly inhibits acid secretion in the frog gastric mucosa. Due to the larger size and lipophilicity, a larger concentration of methylamine was required to inhibit acid secretion as effectively as with NH₄Cl. In addition, acid secretion did not recover fully after inhibition with methylamine because methylamine will not exit cells as rapidly as does NH₄Cl.

Exposure of the gastric mucosa to NH₄Cl does not block acid secretion by blockade of basolateral K⁺-channel activity. Blockade of basolateral K⁺ channel activity does not significantly influence the rate of stimulated acid secretion in the gastric mucosa. One hour or more of incubation with barium (5 mM) is required to significantly reduce the rate of stimulated acid secretion in the frog gastric mucosa and by 3 hr acid secretion was reduced only 44% when compared to control tissues. In addition, inhibition of acid secretion does not occur in 3 hr when the barium concentration is reduced to 0.5 or 1 mM. Incubation of stimulated tissues with barium also caused a significant increase in R with little to no influence on PD. Inhibition of K⁺ channel activity with tolbutamide, clotrimazole, or TEA had no effect on acid secretion, R, or PD, therefore these K⁺ channel blockers are not effective in the frog gastric mucosa.

That an increase in R of the gastric mucosa incubated with NH₄Cl occurs only in stimulated tissues suggests that NH₄Cl acts to specifically inhibit stimulated acid secretion. In addition to numerous basolateral K⁺ channels that recycle K⁺ imported by the Na, K-ATPase, it is thought that oxyntic and parietal cells possess an apical K⁺ channel(s) that is (are) active only during acid secretion. The apical K⁺ channel facilitates the movement of cytoplasmic K⁺ into the gastric lumen; K⁺ is then recycled back into the cell by H, K-ATPase activity during acid secretion (Forte and Reenstra (1994) In: Advances in Comparative and Environmental Physiology. Berlin, Springer-Verlag, Chapt. 13, p. 239-259). The apical K⁺ channel represents an important regulatory site for activity of the gastric H, K-ATPase. NH₄⁺ is a potent inhibitor of Ca²⁺-dependent inwardly rectifying K⁺ channel activity in HeLa cells (Diaz et al. (1996.) Biochim. Biophys. Acta 1284: 119-121). Studies that address the relationship of NH₄⁺ and apical K⁺ channel activity in gastric oxyntic and parietal cells are important and merit further investigation.

Blockage of an apical K⁺ channel by NH₄Cl, leads to the important questions (i) why only ammonia and not other K⁺ channel blockers inhibit acid secretion and (ii) why ammonia (and Ba²⁺) do not effectively block acid secretion when administered from the luminal solution. It is reasonable to believe from the results presented in this invention that NH₄Cl blocks acid secretion from the cytosolic face of the K⁺ channel. Thus, NH₄Cl can get into cells and to the cytosolic aspect of the apical membrane by virtue of its lipophilicity but barium (and other K⁺ channel blockers) cannot. A reasonable conclusion is that barium can effectively block an apical K⁺ conductance when the apical membrane is isolated from the intact cell. The reason why NH₄Cl does not inhibit acid secretion from the apical surface may be due to the lack of permeability of the apical membrane to NH₄Cl (Boron et al. (1994) J. Exp. Biol. 196: 347-360), or may be because the secretory “flush” during acid secretion makes it difficult for substances to interact directly with the apical membrane. Further work is needed to understand in detail, the effects of NH₄Cl on acid secretion in intact tissues.

This invention concerning the action of ammonia on gastric oxyntic cells is not consistent with that set-forth by Fryklund, et al. ((1990) Am. J. Physiol. 258: G719-G727). In that study, aminopyrine uptake, oxygen consumption, and glucose oxidation were shown to increase in isolated and stimulated glands (rabbit) and decrease in inhibited glands. Since glucose oxidation also increased in the presence of NH₄⁺, it was suggested that ammonia stimulates H, K-ATPase activity. It was proposed that NH₃ enters the canalicular membrane, is protonated by secreted H⁺ to form NH₄⁺, and then NH₄⁺ acts as a surrogate for K⁺ on the active H, K-ATPase. Since the flux of a related primary alkylamine (methylamine) is much slower than the measured rate of acid secretion in stimulated tissues, it is unlikely that titration and recycling can account for a rapid and complete inhibition of acid secretion in gastric tissues. The effect of NH₄Cl on R in inhibited and stimulated tissues and the inhibition of acid secretion by related alkylamines that do not interact with the H, K-ATPase, supports the present invention of K⁺ channel activity regulating acid secretion in gastric cells.

As described herein, a method to regulate or modulate acid secretion mediated by an apical, inwardly rectifying K⁺ channel in gastrointestinal cells has been discovered. The method comprises contacting the cells with an agent that regulates the activity of the apical, inwardly rectifying K⁺ channel wherein the activity of the apical, inwardly rectifying K⁺ channel is associated with gastric acid secretion. Regulation or modulation of this activity, as used herein, includes increased acid secretion, or conversely, in another embodiment, the inhibition or reduction of acid secretion. The range of acid secretion inhibition can include complete or partial inhibition of acid secretion. For example, a range of acid secretion can typically be from 1-10%, to 11-50% and up to 51-100%.

In one embodiment of the present invention, the inwardly rectifying K⁺ channel is expressed at the apical surface of gastrointestinal cells, which include, but are not limited to, oxyntic cells and parietal cells.

One embodiment of this invention pertains to acid secretion mediated by an apical, inwardly rectifying K⁺ channel which comprises Kir4.1, wherein the K⁺ channel is homomeric for Kir4.1. Furthermore, the apical, inwardly rectifying K⁺ channel can comprise heteromeric Kir4.1. Other proteins can be associated with the K⁺ channel of the present invention. For example, Kir 5.1, Kir 4.2 or Kir1.1 proteins can also be associated with the channels described herein.

Accordingly, in one embodiment of this invention, the agent that regulates acid secretion mediated by an apical, inwardly rectifying K⁺ channel is selected from the group consisting of, but not limited to, a small molecule inhibitor or antagonist and a weak base inhibitor or antagonist, whereby acid secretion is modulated by the agent. The term “inhibitor” or “antagonist” as used herein is an agent that reduces the function or biological activity of a molecule partially or completely.

The invention also provides a method (also referred to herein as a “screening assay”) for identifying modulators, i.e., candidate or test compounds or agents (e.g., antisense, polypeptides, peptidomimetics, small molecules or other drugs) which bind to the apical, inwardly rectifying K⁺ channel described herein or have a stimulatory or inhibitory effect on, for example, the activity of gastric acid secretion mediated by an apical, inwardly rectifying K⁺ channel.

In one embodiment, the invention provides assays for screening candidate or test compounds which modulate the activity of gastric acid secretion mediated by an apical, inwardly rectifying K⁺ channel. The test compounds to be screened in the methods of the present invention can be designed using numerous methods known to those of skill in the art. For example, one can use any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam (1997) Anticancer Drug Des. 12:145).

Examples of methods for the synthesis of molecular libraries can be found in the art, for example in DeWitt et al. (1993) Proc. Natl. Acad. Sci. U.S.A., 90:6909; Erb et al. (1994) Proc. Natl. Acad. Sci. U.S.A., 91:11422; Zuckennann et al. (1994). J. Med. Chem., 37:2678; Cho et al. (1993) Science, 261:1303; Carell et al. (1994) Angew, Chem. Int. Ed. Engl., 33:2059; Carell et al. (1994) Angew. Chem. Int. Ed. Engi., 33:2061; and Gallop et al. (1994) J. Med. Chem., 37:1233.

Libraries of compounds may be presented in solution (e.g., Houghten (1992) Biotechniques, 13:412-421), or on beads (Liam (1991) Nature, 354:82-84), chips (Fodor (1993) Nature, 364;555-556), bacteria (Ladner U.S. Pat. No. 5,223,409), spores (Ladner U.S. Pat. No. '409), plasmids (Cull et al. (1992) Proc. Natl. Acad. Sci. USA, 89:1865-1869) or on phage (Scott and Smith (1990) Science, 249:386-390; Devlin (1990) Science, 249:404-406; Cwirla et al. (1990) Proc. Natl. Acad. Sci. USA, 97:6378-6382; Felici (1991) J. Mol. Biol., 222:301-310; Ladner Id.).

In one embodiment, an assay is a cell-based assay in which a cell which expresses an apical, inwardly rectifiing K⁺ channel, is contacted with a test compound and the ability of the test compound to bind to or interact with the apical, inwardly rectifying K⁺ channel is determined. The cell, for example, can be a parietal cell. Determining the ability of the test compound to bind to the apical, inwardly rectifying K⁺ channel can be accomplished, for example, by coupling the test compound with a radioisotope or enzymatic label such that binding of the test compound to the receptor can be determined by detecting the labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

It is also within the scope of this invention to determine the ability of a test compound to interact with the apical, inwardly rectifying K⁺ channel without the labeling of any of the interactants. For example, a microphysiometer can be used to detect the interaction of a test compound with the apical, inwardly rectifying K⁺ channel without the labeling of either the test compound or the apical, inwardly rectifying K⁺ channel. McConnell et al. (1992) Science, 257:1906-1912. As used herein, a “microphysiometer” (e.g., Cytosensor™) is an analytical instrument that measures the rate at which a cell acidifies its environment using a light-addressable potentiometric sensor (LAPS). Changes in this acidification rate can be used as an indicator of the interaction between the agent and the apical, inwardly rectifying K⁺ channel.

In another embodiment, an assay is a cell-based assay comprising contacting a cell expressing an apical, inwardly rectifying K⁺ channel, that mediates acid secretion as described herein, with a test compound and determining the ability of the test compound to modulate or alter (e.g., stimulate or inhibit) the activity of the apical, inwardly rectifying K⁺ channel. Determining the ability of the test compound to modulate the activity of the apical, inwardly rectifying K⁺ channel can be accomplished, for example, by determining the quantity of acid secretion.

This invention further pertains to novel agents identified by the above-described screening assays. Accordingly, it is within the scope of this invention to further use an agent identified as described herein in an appropriate animal model. For example, an agent identified as described herein (e.g., a modulating agent, an antisense nucleic acid molecule, a specific antibody, or a protein-binding partner) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

The present invention provides for both prophylactic and therapeutic methods of treating a subject at risk of (or susceptible to) a disorder or having a disorder associated with aberrant expression or activity of an apical, inwardly rectifying K⁺ channel that mediates acid secretion.

Prophylactic Methods

In one aspect, the invention provides a method for preventing in a subject, an acid-secreting disease or condition associated with the aberrant expression or activity of genes or proteins which comprise the K⁺ channel of the present invention, by administering to the subject an agent which modulates expression of a protein comprising the apical, inwardly rectifying K⁺ channel, or at least one activity of the apical, inwardly rectifying K⁺ channel that mediates acid secretion. Subjects that have increased acid secretion would be administered an agent which is an antagonist for the apical, inwardly rectifying K⁺ channel that mediates acid secretion, whereas subjects that have decreased acid secretion would be administered an agent which is an agonist of the apical, inwardly rectifying K⁺ channel that mediates acid secretion. Subjects at risk for a disease which is caused or contributed to by aberrant gene expression or protein activity can be identified by, for example, gastric acid secretion. Such tests for acid secretion are well-known to those of shill in the art. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

Therapeutic Methods

Another aspect of the invention pertains to methods of modulating expression or activity of the gastric acid secretion mediated by an apical, inwardly rectifying K⁺ channel of the invention for therapeutic purposes. The modulatory method of the invention involves contacting a cell with an agent that modulates one or more of the activities of the apical, inwardly rectifying K⁺ channel that mediates acid secretion. An agent that modulates protein activity can be an agent as described herein, such as a nucleic acid or a protein, a naturally-occurring target molecule of a protein described herein, a polypeptide, a peptidomimetic, a weak base inhibitor or other small molecule. In one embodiment, the agent stimulates one or more protein activities. Examples of such stimulatory agents include active protein as well as a nucleic acid molecule encoding the protein that has been introduced into the cell. In another embodiment, the agent inhibits one or more protein activities. Examples of such inhibitory agents include antisense nucleic acid molecules, weak base inhibitors, small molecule inhibitors and anti-protein antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of an apical, inwardly rectifying K⁺ channel that mediates acid secretion. In one embodiment, the method involves administering an agent (e.g., an agent identified by a screening assay described herein), or combination of agents that modulates (e.g., upregulates or downregulates) expression or activity of an apical, inwardly rectifying K⁺ channel that mediates acid secretion. In another embodiment, the method involves administering a protein or nucleic acid molecule of the invention as therapy to compensate for reduced or aberrant expression or activity of the apical, inwardly rectifying K⁺ channel that mediates acid secretion.

Stimulation of protein activity is desirable in situations in which the protein is abnormally downregulated and/or in which increased protein activity is likely to have a beneficial effect. Likewise, inhibition of protein activity is desirable in situations in which the protein is abnormally upregulated and/or in which decreased protein activity is likely to have a beneficial effects. Accordingly, disorders associated with acid secretion mediated by an apical, inwardly rectifying K⁺ channel can be selected from the group consisting of, but not limited to, gastric ulcers, duodenal ulcers, gastritis, duodenitis, and reflux esophagitis. In a further embodiment of this invention, the disorder associated with acid secretion mediated by an apical, inwardly rectifying K⁺ channel is associated with Helicobacter pylori infection.

### EXAMPLES

### Example 1

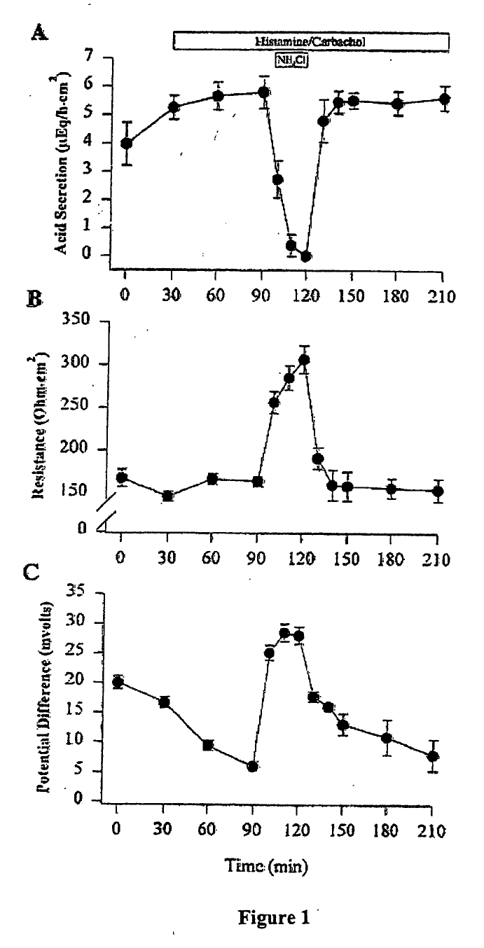

Nutrient ammonium chloride (10 mM) rapidly and reversibly inhibits stimulated acid secretion. Acid secretion in frog fundic mucosa attained a maximal rate of 5.80±0.1 2 μEq/hr cm² by 90 min after stimulation (FIG. 1A).

While R remained constant at 164.7±5.61 Ohm cm² (FIG. 1B), PD declined slowly to 6.00±0.70 mvolts by 90 min after stimulation (FIG. 1C). Similar results were obtained after stimulation with forskolin.

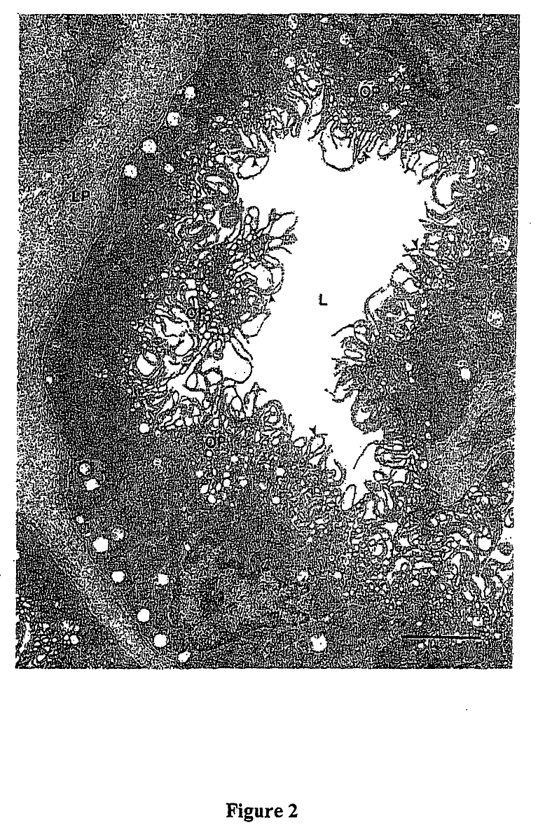

When 10 mM NH₄Cl was added to the nutrient solution (pH 7.2) of tissues stimulated with either histamine/carbachol (FIG. 1A) or forskolin, the rate of acid secretion declined to 0±0 μEq/hr cm² within 30 min. Although incubation of stimulated mucosa with 1-9 mM NH₄Cl resulted in an inhibition of acid secretion, 10 mM NH₄Cl was the least required to result in complete inhibition of acid secretion within 30 min. Concomitant with a rapid reduction in acid secretion in the presence of 10 mM NH₄Cl, R and PD increased significantly to 306.5±16.11 Ohm cm² and 28.48±1.47 mvolts, respectively. Even though acid secretion was completely inhibited in 30 min by 10 mM NH₄Cl, tissue morphology at this time point showed oxynticopeptic cells in a stimulated rather than inhibited configuration (FIG. 2). The rate at which acid secretion declined over time in the presence of 10 mM NH₄Cl was 3.6 times faster than with cimetidine or 3.2 times faster than with omeprazole (see Table).

TABLE
Rate of Acid Secretion in the Presence of
Cimetidine, Omeprazole, NH₄Cl,
or KCN.

| | Acid | | | Rate of decline in |
|---|---|---|---|---|
| | secretion | Time | Correlation | acid secretion |
| | (μEq/h · cm²) | (min) | coefficient | (μEq/h · cm²)/min |
| Cimetidine | 5.59 ± 0.13 | 90″ | −0.997 | −0.07935 |
| | 3.51 ± 0.21 | 120″ | | |
| | 0.83 ± 0.22 | 150″ | | |
| Omeprazole | 5.45 ± 0.30 | 90″ | −0.989 | −0.09034 |
| | 2.03 ± 0.21 | 120″ | | |
| | 0.30 ± 0.10 | 150″ | | |
| Ammonium | 5.80 ± 0.12 | 90″ | −0.999 | −0.2881 |
| chloride | 2.73 ± 0.14 | 100″ | | |
| (NH₄Cl) | 0.38 ± 0.08 | 110″ | | |
| Potassium | 5.63 ± 0.12 | 90″ | −0.998 | −0.2422 |
| cyanide | 3.03 ± 0.25 | 100″ | | |
| (KCN) | 0.864 ± 0.15 | 110″ | | |

Values are means±SEM. Tissues from the frog gastric mucosa were stimulated for 90 min with histamine/carbachol, washed, and then incubated for 180 min with 1 mM cimetidine or 0.3 mM omeprazole or for 30 min with 10 mM NH₄Cl or 2 mM KCN. The rate at which acid secretion declined over time was determined from the linear portion of the inhibition curve for each compound. Data were obtained from 6-7 experiments (cimetidine, omeprazole), 21 experiments (NH₄Cl), or 6 experiments (KCN).

When 10 mM NH₄Cl was washed from the nutrient bath and tissues incubated in buffer without NH₄Cl, acid secretion, R, and PD returned to near-normal values of 5.51±0.16 μEq/hr cm², 159.0±16.83 Ohm cm², and 7.95±2.65 mvolts, respectively within 90 min (FIGS. 1A-C).

### Example 2

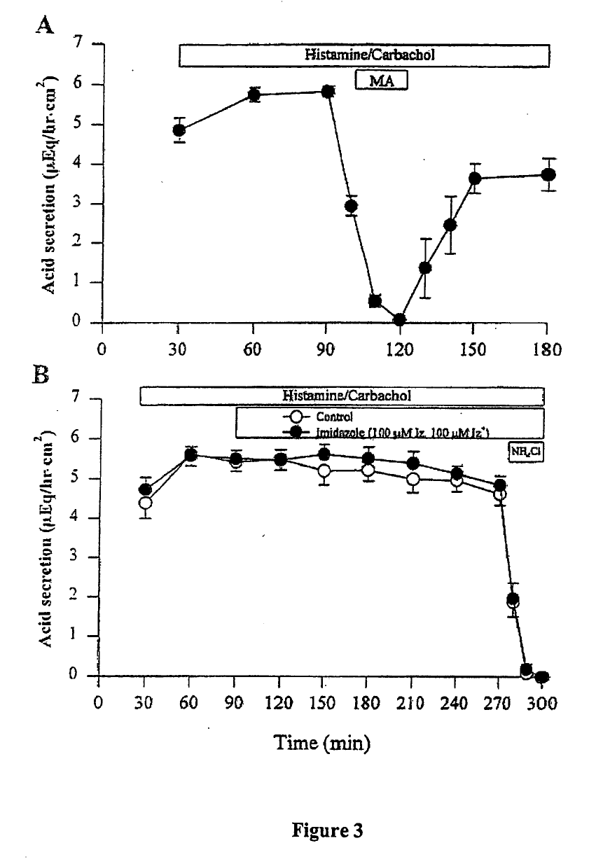

Acid secretion is rapidly inhibited by methylamine but not by imidazole. Methylamine and imidazole were used to determine whether other small primary alkylamines or weak bases, respectively, inhibit acid secretion (FIG. 3). Methylamine inhibited stimulated acid secretion from 5.81±0.125 μEq/hr cm² to 0.081±0.056 μEq/hr cm² within 30 min at a minimum concentration of 50 mM (FIG. 3A). However, when methylamine was washed from the nutrient solution, acid secretion returned to only 60% (3.74±0.41 μEq/hr cm²) of the starting level within 60 min (FIG. 3A). When imidazole was used at a concentration similar to that of NH₃ in the nutrient buffer (100 μM Iz versus 92.5 μM NH₃), acid secretion decreased slowly from 5.47±0.26 to 4.86±0.29 μEq/hr cm² in 3 hr (180 min). This reduction in acid secretion over time was not significantly different from that of control tissues (FIG. 3B). Increasing the concentration of imidazole 50-fold to 10 mM (5 mM Iz, 5 mM Iz⁺) decreased acid secretion rapidly from 6.60±0.43 to 4.60±0.43 μEq/hr cm² in 30 min and to 4.06±0.46 μEq/hr cm² in 3 hr. When 10 mM nutrient NH₄Cl was added to imidazole-treated tissues, acid secretion was inhibited to 0±0 μEq/hr cm² within 30 min (FIG. 3B).

### Example 3

Serosal to mucosal flux of ammonia and methylamine is not great enough to neutralize secreted H⁺. To determine whether nutrient 10 mM NH₄Cl or 50 mM methylamine block acid secretion by moving rapidly from nutrient to luminal solutions, and thus neutralizing secreted H⁺, the flux of ammonia (NH₃) and methylamine was measured.

After addition of 10 mM NH₄Cl to the nutrient solution, the ammonia (NH₃) concentration was 599.76 μM. At the start, NH₃ was not detectable in the luminal solution. By 30 min after addition of 10 mM NH₄Cl to the nutrient solution, the nutrient NH₃ concentration was reduced to 538.3±6.7. Thus, 61.46 μM of NH₃ was lost from the nutrient solution. However, luminal NH₃ concentration after 30 min increased in only one group (n=4) resulting in a mean concentration of 0.00133±0.00133 μM. These results suggest that the NH₃ lost from the nutrient buffer within 30 min reside in the cells and intracellular spaces of the tissue.

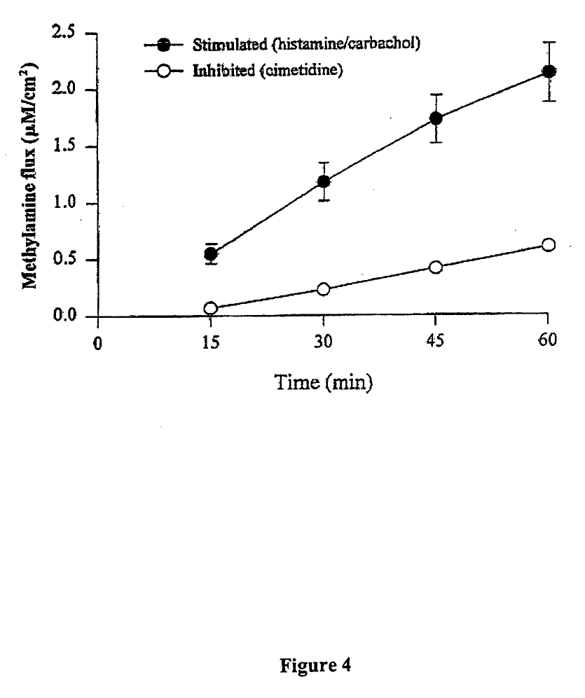

It is possible, however, that ammonia cannot be detected in the luminal solution of stimulated tissues because the forward movement of NH₃ is balanced by the backward movement of NH₄⁺ into the cell by the H, K-ATPase, as has been described (Fryklund et al. (1990) Am. J. Physiol. 258: G719-G727). Thus, serosal to mucosal flux studies with methylamine, a compound that should not interact with the H, K-ATPase, were performed (FIG. 4).

The rate of methylamine flux in inhibited tissues was 0.601±0.114 μM/h cm² and was 2.25±0.088 μM/h cm² in stimulated tissues (FIG. 4). The rate of acid secretion was 0.099±0.089 μEq/h cm² in inhibited tissues and 7.0±0.3 μEq/h cm² in stimulated tissues. As it would require 7.0 μM/h cm² of methylamine in the luminal solution to titrate all of the H⁺ generated during acid secretion, our data demonstrate that titration alone cannot account for the total inhibition of acid secretion that occurs with methylamine. Rather, acid secretion would decline slowly over time, a situation similar to results with other weak bases such as imidazole.

### Example 4

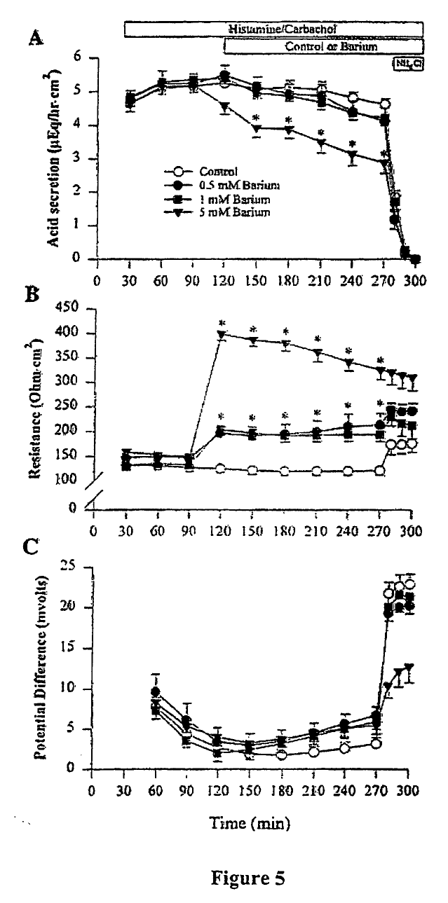

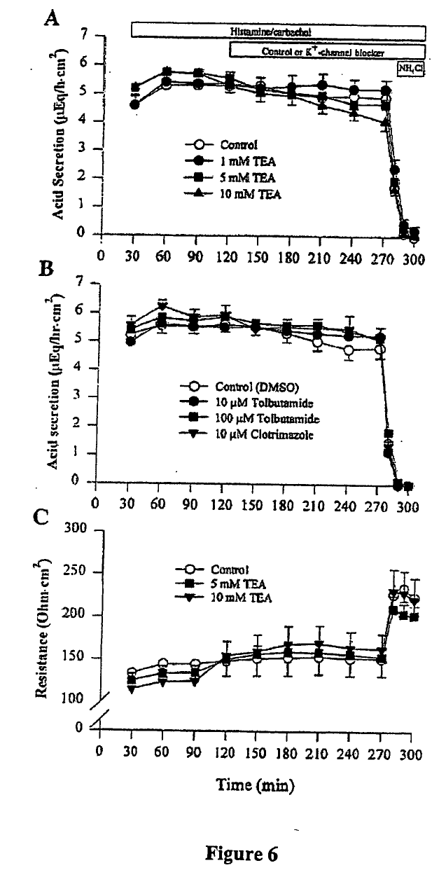

Blockade of basolateral K⁺ channel activity does not mitigate the inhibitory effects of NH₄Cl on stimulated acid secretion. Barium at 0.5 and 1 mM reduced acid secretion in 3 hr (180 min) from 5.23±0.29 and 5.35±0.22 μEq/hr cm² to 4.12±0.38 and 4.20±0.21 μEq/hr cm², respectively (FIG. 5A). This decline in the rate of acid secretion over time was not significantly different from that of control tissues (FIG. 5A). In contrast, incubation of stimulated tissues with 5 mM barium decreased the rate of acid secretion from 5.19±0.22 to 2.89±0.33 μEq/hr cm² in 3 hr (180 min). This reduction in the rate of acid secretion was significantly different from that of control tissues (FIG. 5A). Acid secretion in the presence of another wide-spectrum K⁺ channel blocker such as TEA (FIG. 6A), tolbutamide which blocks ATP-dependent K⁺ channel activity (FIG. 6B) or clotrimazole which blocks Ca²⁺- and cAMP-dependent K⁺ channel activity (FIG. 6B) showed no significant decline when compared to control tissues. When tissues treated with 0.5-5 mM barium, 1-10 mM TEA, 10-100 μM tolbutamide, or 10 μM clotrimazole were incubated with 10 mM NH₄Cl, acid secretion was inhibited to 0 μEq/hr cm² within 30 min in all groups (FIGS. 5 and 6).

Addition of 0.5, 1, or 5 mM barium to the nutrient solution resulted in a significant increase in R in all groups (FIG. 5B). Whereas the largest increase in R occurred in tissues incubated with 5 mM barium, tissues incubated with 0.5 and 1 mM barium also showed a significant increase in R when compared to control tissues (FIG. 5B). When tissues were treated with TEA (FIG. 6C), tolbutamide, or clotrimazole, the mean R was not significantly different from that of control tissues. Tissues incubated with 0.5 and 1 mM barium (FIG. 5B), TEA (FIG. 6C), tolbutamide, or clotrimazole and 10 mM NH₄Cl showed an increase in R within 30 min.

Whereas the mean PD of tissues incubated with barium was increased, this increase was not significantly different from that of control tissues (FIG. 5C). Similar results were obtained for tissues incubated with TEA, tolbutamide, or clotrimazole. Tissues incubated with barium (FIG. 5C), TEA, tolbutamide, or clotrimazole and 10 mM NH₄Cl showed an increase in PD within 30 min.

### Example 5

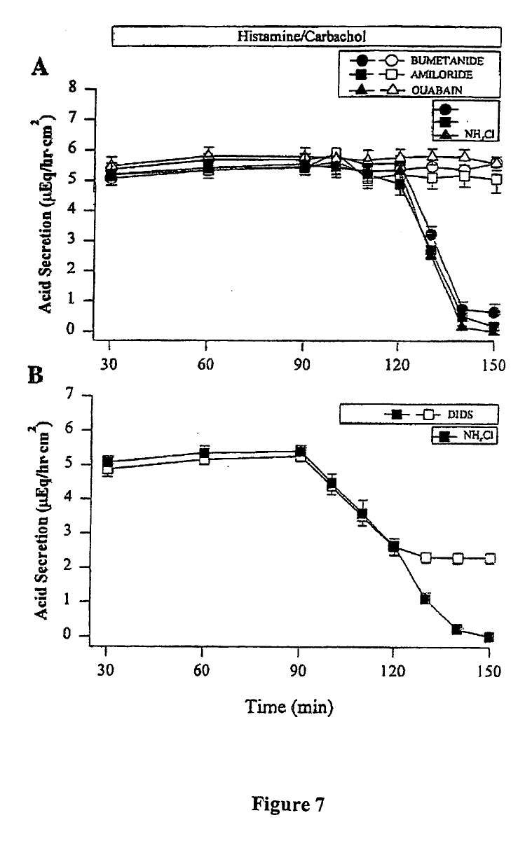

Blockade of basolateral transporter activity does not significantly) abrogate the ammonia-induced inhibition of acid secretion. Incubation of stimulated tissues with 100 μM bumetamide, 1 mM amiloride, or 1 mM oubain for 60 min caused no significant reduction in acid secretion (FIG. 7A). In contrast, acid secretion was reduced from 5.25±0.12 to 2.29±0.18 μEq/hr cm² in stimulated tissues incubated with 0.3 mM DIDS for 60 min (FIG. 7B). When stimulated tissues were incubated for 30 min with one of the 4 ion transport blockers and then further incubated with 10 mM NH₄Cl, acid secretion declined to 0.645±0.278 μEq/hr cm² (bumetanide), 0.188±0.140 μEq/hr cm² (amiloride), 0 μEq/hr cm² (oubain), or 0 μEq/hr cm² (DIDS) within 30 min (FIGS. 7A, B). Similar results were obtained when a combination of bumetamide, amiloride, and oubain (followed by NH₄Cl) was added to stimulated tissues.

### Example 6

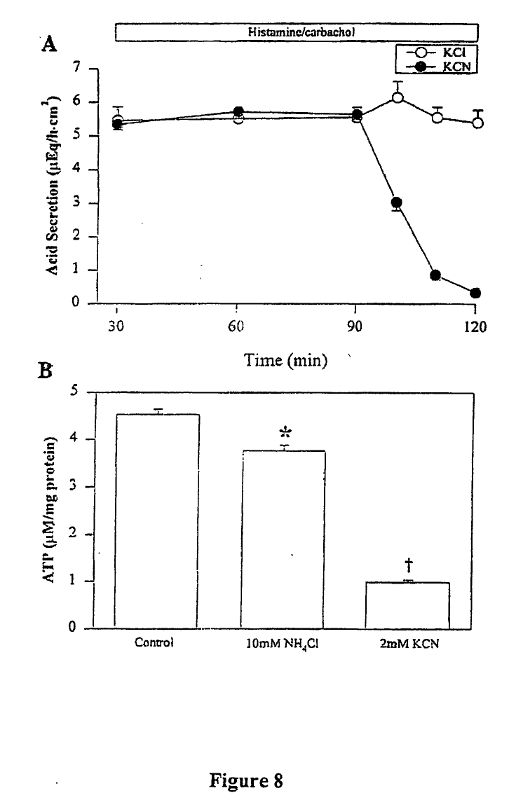

KCN inhibits stimulated acid secretion like 10 mMNH₄Cl but results in significantly greater depletion of intracellular ATP. Acid secretion in the frog gastric mucosa was inhibited in a dose-dependent manner after addition of the metabolic inhibitor, potassium cyanide (KCN). Whereas 250 PM KCN significantly reduced acid secretion, 2 mM KCN was required to inhibit acid secretion to 0.33±0.04 μEq/hr cm² within 30 min (FIG. 8A). The rate of reduction of acid secretion with 2 mM KCN was, like 10 mM NH₄Cl, 3 times faster than with cimetidine or omeprazole (see Table). Inhibition of acid secretion with 2 mM KCN was accompanied by an increase in R from 112.4±8.16 to 613.7±38.19 Ohm cm² and potential difference from 4.72±2.59 to 15.05±1.20 mvolts.

To determine whether 10 mM NH₄Cl reduced intracellular ATP like 2 mM KCN we prepared gastric glands from the rabbit and measured intracellular ATP. In resting glands, 2 mM KCN significantly reduced intracellular ATP within 30 min to 0.982±0.051 μM/mg protein (21.2% of control) whereas 10 mM NH₄Cl decreased intracellular ATP to 3.76±0.12 μM/mg protein (83% of control, FIG. 8B).

### Example 7

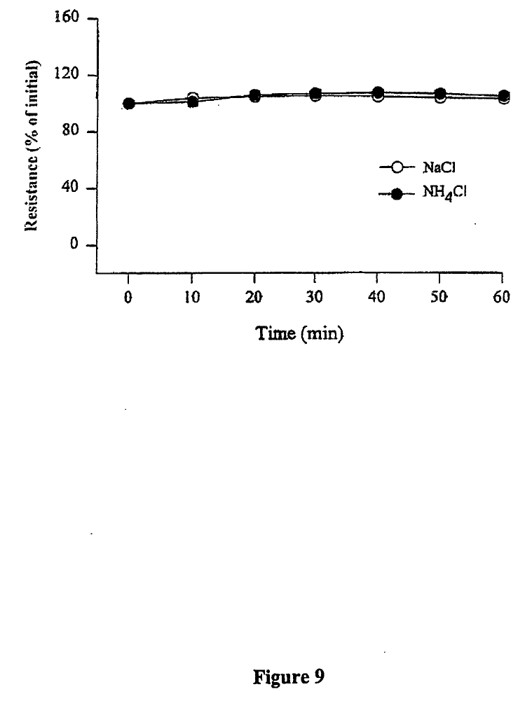

NH₄Cl does not increase the R of cimetidine-inhibited tissues. Tissues were incubated with cimetidine until acid secretion reached 0 μEq/hr cm²; experiments were performed only if the R of paired tissues was different by 10% or less. When 10 mM NH₄Cl was added to the nutrient solution of cimetidine-inhibited tissues (pH 7.2), there was no change in R when compared to control tissues incubated with NaCl (FIG. 9). These results are in contrast to those in tissues stimulated with histamine/carbachol (FIG. 1B), where tissue R increased significantly in the presence of 10 mM NH₄Cl. In addition, when K⁺ channel activity in cimetidine-inhibited tissues was blocked with either 1 mM or 5 mM barium, R increased to 126.0±5.0% and 140.0±7.0% respectively, of the initial R.

The relevant teachings of all the references, patents and/or patent applications cited herein are incorporated by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A method to regulate acid secretion mediated by an apical, inwardly rectifying K⁺ channel in gastrointestinal cells, comprising contacting the cells with an agent that regulates the activity of the apical, inwardly rectifying K⁺ channel, thereby regulating acid secretion.

2. The method according to claim 1 wherein the acid secretion is stimulated acid secretion.

3. The method according to claim 1 wherein the gastrointestinal cells are oxyntic cells.

4. The method according to claim 1 wherein the gastrointestinal cells are parietal cells.

5. The method according to claim 1 wherein the apical, inwardly rectifying K⁺ channel comprises Kir 4.1.

6. The method according to claim 1 wherein the apical, inwardly rectifying K⁺ channel comprises Kir 4.1, and further comprises one or more proteins selected from the group consisting of Kir 4.2, Kir 5.1 and Kir 1.1.

7. The method according to claim 1 wherein the regulation results in the inhibition of acid secretion by gastrointestinal cells.

8. The method according to claim 7 wherein acid secretion by gastrointestinal cells is inhibited by administration of an antagonist of the inwardly rectifying K⁺ channel.

9. The method according to claim 8 wherein the antagonist is a small molecule inhibitor.

10. The method according to claim 8 wherein the antagonist is a weak base inhibitor.

11. A method to regulate the activity of an apical, inwardly rectifying K⁺ channel, wherein the activity is associated with acid secretion, comprising contacting the apical, inwardly rectifying K⁺ channel with an agent that alters the activity of the apical, inwardly rectifying K⁺ channel, thereby regulating the activity of the apical, inwardly rectifying K⁺ channel.

12. The method according to claim 11 wherein the apical, inwardly rectifying K⁺ channel comprises Kir 4.1.

13. The method according to claim 11 wherein the apical, inwardly rectifying K⁺ channel comprises Kir 4.1, and further comprises one or more proteins selected from the group consisting of Kir 4.2, Kir 5.1 and Kir 1.1.

14. The method according to claim 11 wherein the activity of an apical, inwardly rectifying K⁺ channel is inhibited.

15. The method according to claim 11 wherein the activity of an apical K⁺ channel is inhibited by administration of an antagonist.

16. The method according to claim 15 wherein the antagonist is a small molecule inhibitor.

17. The method according to claim 15 wherein the antagonist is a weak base inhibitor.

18. A method to screen for agents that regulate acid secretion comprising assaying the quantity of acid secretion mediated by an apical, inwardly rectifying K⁺ channel from parietal cells or oxyntic cells stimulated to secrete acid, in the presence and absence of the agent to be tested, wherein reduced acid secretion in the presence of the agent, as compared to the acid secretion in the absence of the agent, indicates that the agent is an antagonist of acid secretion.

19. The method according to claim 18 wherein the apical, inwardly rectifying K⁺ channel comprises Kir 4.1.

20. The method according to claim 18 wherein the apical, inwardly rectifying K⁺ channel comprises Kir 4.1, and further comprises one or more proteins selected from the group consisting of Kir 4.2, Kir 5.1 and Kir 1.1.

21. A method to treat an individual with a disorder associated with gastric acid secretion mediated by an apical, inwardly rectifying K⁺ channel comprising administering a therapeutically effective amount of an agent which regulates the activity of the apical, inwardly rectifying K⁺ channel.

22. The method according to claim 21 wherein the disorder is selected from the group consisting of gastric ulcers, duodenal ulcers, gastritis, duodenitis, and reflux esophagitis.

23. The method according to claim 21 wherein the disorder is associated with Helicobacter pylori infection.

24. The method according to claim 21 wherein the apical, inwardly rectifying K⁺ channel comprises Kir 4.1.

25. The method according to claim 21 wherein the apical, inwardly rectifying K⁺ channel comprises Kir 4.1, and further comprises one or more proteins selected from the group consisting of Kir 4.2, Kir 5.1 and Kir 1.1.

26. The method according to claim 21 wherein the agent is an antagonist of the apical, inwardly rectifying K⁺ channel.

27. The method according to claim 26 wherein the antagonist is a small molecule inhibitor.

28. The method according to claim 26 wherein the antagonist is a weak base inhibitor.

29. Use of an agent which regulates an apical, inwardly rectifying K⁺ channel for the manufacture of a medicament for the treatment of a disorder associated with gastric acid secretion mediated by the apical, inwardly rectifying K⁺ channel in an individual.
